(19) United States
(12) Patent Application Publication
(10) Pub. No.: US 2018/0134730 A1
(43) Pub. Date: May 17, 2018

(54) IMPROVED PROCESS FOR PREPARING BORYL 7-AZAINDOLE COMPOUNDS

(71) Applicant: BoroPharm Inc., Novi, MI (US)

(72) Inventors: Sarah Caruso, Novi, MI (US); Matthew Forster, Novi, MI (US); Andrew Cipa, Novi, MI (US); Mark Marlatt, Novi, MI (US); Todd Zahn, Novi, MI (US); Paul Herrinton, Novi, MI (US)

(21) Appl. No.: 15/576,164

(22) PCT Filed: May 9, 2016
(86) PCT No.: PCT/US16/31436

§ 371 Date: Nov. 21, 2017

Related U.S. Application Data
(60) Provisional application No. 62/166,511, filed on May 26, 2015.

(51) Int. Cl. C07F 5/02; A61K 31/69

(52) U.S. Cl. CPC C07F 5/025; A61K 31/69

(57) ABSTRACT

The present disclosure relates to an improved process for the preparation of 3-boryl substituted azaindole compounds in high yields and high selectivity. Such azaindole compounds may be used as intermediates to form compounds with cytotoxic, anticancer, and antiviral activities.

This application claims the benefit of U.S. Provisional Application No. 62/166,511, filed May 26, 2015, which is incorporated by reference in its entirety.

### FIELD OF THE INVENTION

The present invention relates to an improved process for the preparation of boryl 7-azaindole compounds, such as 3-boryl substituted 7-azaindole compounds in high yields and high selectivity.

### BACKGROUND OF THE INVENTION

U.S. Pat. No. 8,829,007 discloses compounds that inhibit the replication of influenza viruses, including (2S,3S)-3-((5-fluoro-2-(5-fluoro-1H-pyrrolo[2,3-b]pyridin-3-yl)pyrimidin-4-yl)amino)bicyclo[2.2.2]octane-2-carboxylic acid (also known as VX-787). Boroylated intermediates are useful for preparing these compounds that inhibit the replication of influenza viruses. M. P. Clark et al., J. Med. Chem., 2014, 57-6668-6678. These borylated intermediates were previously prepared by incorporating a bromine at the position of the molecule to be borylated. For example, Clark reports preparing 5-chloro-3-(4,4,5,5-tetramethyl-1,3,2-dioxaborolan-2-yl)-1-tosyl-1H-pyrrolo[2,3-b]pyridine from 3-bromo-5-fluoro-1H-pyrrolo[2,3-b]pyridine.

Methods for preparing borylated compounds are described in U.S. Patent Publication Nos. 2008/0146814 and 2008/0167476.

Improved methods for preparing 3-boryl 7-azaindole compounds, such as 3-boryl-5-halo-7-azaindole compounds, in high yield and with no or few impurities are needed.

### SUMMARY OF THE INVENTION

The present invention relates to improved processes for preparing 3-boryl substituted 7-azaindole compounds, such as 3-boryl 5-halo 7-azaindole compounds. The present inventors discovered that borylation of 7-azaindole compounds can occur at both the 3- and 4-positions of the azaindole ring as well as other parts of the compound leading to reduced yields and the need for additional purification procedures. In some instances, separation of the 3- and 4-position isomers (such as 5-fluoro-7-azaindole substituted at the 3- or 4-position with 4,4,5,5-tetramethyl-1,3,2-dioxaborolan-2-yl) is difficult. The processes described herein can produce 3-boryl compounds with greater selectivity over other borylated isomers, such as 4-boryl compounds.

In one aspect, the present invention relates to a process for preparing a compound of formula (I):

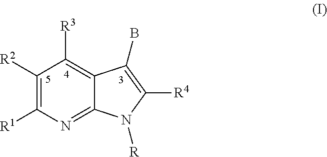

wherein

R is hydrogen or a nitrogen protecting group;

R¹, R², R³ and R⁴ are each independently selected from hydrogen, halogen, hydroxyl, nitro, amino, substituted or unsubstituted alkylamino, substituted or unsubstituted dialkylamino, cyano, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted cycloalkylalkyl, substituted or unsubstituted heterocyclylalkyl, substituted or unsubstituted heteroaryl, substituted or unsubstituted arylalkyl, and substituted or unsubstituted heteroarylalkyl; and

B is a boryl containing group where the boron atom of the group is attached to the carbon ring atom at the 3-position of the azaindole. In one embodiment, the process comprises reacting a compound of formula (II)

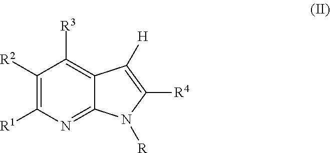

with a boron containing compound in the presence of an organic ligand and a catalyst selected from an iridium catalyst, a rhodium catalyst, a ruthenium catalyst, and any combination thereof.

In one embodiment, the catalyst is an iridium catalyst.

In one embodiment, R is H.

In another embodiment, R is a nitrogen protecting group. For example, in one embodiment, R is selected from tert-butoxycarbonyl, (benzyloxy)carbonyl, N,N-dimethylaminosulfonyl, N,N-dimethylcarboxamide, para-toluenesulfonyl, 9H-fluoren-9-ylmethyloxycarbonyl, a dialkoxyborane (e.g., Pin or a catechol borane), benzenesulfonyl, t-butyl, and t-butyldimethylsilyl (TBDMS). In one preferred embodiment, R is para-toluenesulfonyl.

In one embodiment, R¹, R², R³ and R⁴ are each, independently, selected from hydrogen and halogen. In another embodiment, R¹, R³ and R⁴ are hydrogen and R² is a halogen, such as chloro or fluoro. In one preferred embodiment, R¹, R³ and R⁴ are hydrogen and R² is fluoro.

In one embodiment, R is hydrogen and R¹, R², R³ and R⁴ are each, independently, selected from hydrogen and halogen. In another embodiment, R, R¹, R³ and R⁴ are hydrogen and R² is a halogen, such as chloro or fluoro. In one preferred embodiment, R, R¹, R³ and R⁴ are hydrogen and R² is fluoro.

In one embodiment, R is a nitrogen protecting group (such as para-toluenesulfonyl) and R¹, R², R³ and R⁴ are each, independently, selected from hydrogen and halogen. In another embodiment, R is a nitrogen protecting group (such as para-toluenesulfonyl), R¹, R³ and R⁴ are hydrogen and R² is a halogen, such as bromo, chloro or fluoro. In one preferred embodiment, R is a nitrogen protecting group (such as para-toluenesulfonyl), R¹, R³ and R⁴ are hydrogen and R² is fluoro.

In yet another embodiment, R¹, R², R³ and R⁴ are hydrogen.

In yet another embodiment, R, R¹, R², R³ and R⁴ are hydrogen.

In yet another embodiment, R² is other than hydrogen (e.g., R² is halogen, such as F, Cl, or Br). In one preferred embodiment, R² is fluoro.

In the synthesis of complex molecules containing heteroatom-hydrogen bonds, it is often necessary to selectively protect and deprotect these reactive entities to prevent undesired reactions. Typically, protection involves replacing a reactive hydrogen with a more robust group, while deprotection involves cleaving the robust group and replacing it with hydrogen. The use and removal of protecting groups, however, requires additional synthetic steps, which may reduce product yield and add to the cost of synthesizing. The present inventors discovered that selective borylation at the 3-position of a 7-azaindole (such as a 5-fluoro-7-azaindole) can be performed without protecting the nitrogen ring atom at the 1-position (i.e., R in formula (I) can be hydrogen).

In one embodiment, the reaction is conducted at a temperature of between about 20° C. and about 200° C., such as between about 20° C. and about 150° C., between about 50° C. and about 150° C., between about 50° C. and about 100° C., between about 60° C. and about 80° C. or between about 90° C. and about 110° C. In a preferred embodiment, the reaction is conducted at a temperature of between about 50° C. and about 100° C.

Suitable solvents include, but are not limited to, aliphatic hydrocarbons (such as hexane and heptane), ethers (such as tetrahydrofuran, dioxane, and methyl t-butyl ether), chlorinated alkanes (e.g., dichloromethane and dichloroethane), and any combination thereof. In one embodiment, the reaction is conducted in a solvent selected from hexane, heptane, tetrahydrofuran, and any combination thereof. For example, the reaction may be conducted in a solvent selected from hexane, heptane, and any combination thereof. In another embodiment, the reaction is conducted in tetrahydrofuran.

Suitable catalysts include, iridium catalysts, rhodium catalysts, and ruthenium catalysts. Suitable iridium catalysts include, but are not limited to, [Ir(OMe)(COD)]₂, [Ir(Cl)(COD)]₂, (COD)(η⁵-indenyl)Ir, and any combination thereof. (The abbreviation COD refers to 1,5-cyclooctadiene.) In one preferred embodiment, the catalyst is [Ir(Cl)(COD)]₂.

In one embodiment, the boron containing compound is selected from HBPin, B₂(Cat)₂ diborane and B₂Pin₂, the structures of which are shown below. In a preferred embodiment, the boron containing compound is selected from HBPin and B₂Pin₂.

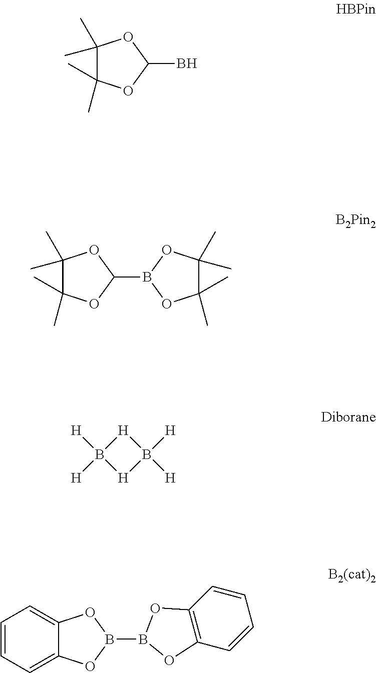

Suitable organic ligands include, but are not limited to, phosphorous containing organic ligands, organic amines, organic imines, ethers, and any combination thereof.

In one embodiment, the organic ligand is selected from those listed below:

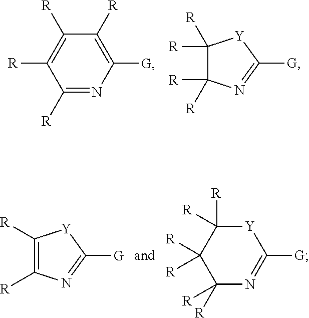

wherein

each occurrence of Y is, independently, CH₂, CHR⁵, CR⁵R⁶, O, S, NH or NR⁵; each occurrence of R is, independently, hydrogen or alkyl (e.g., C₁₋₈ alkyl); each occurrence of R⁵ and R⁶ is independently alkyl (e.g., C₁₋₈ alkyl); and each occurrence of G is, independently, a heteroatom containing group (for example, an O, S, or N-containing group), a heteroatom containing multiple atom chain, or a heteroatom containing multiple atom ring.

For example, in another embodiment, the organic ligand is selected from those listed below:

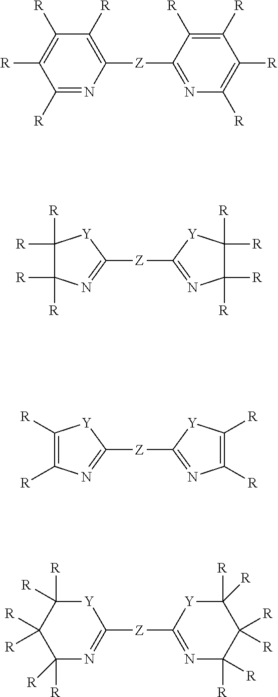

wherein

each occurrence of Y is, independently, CH₂, CHR⁵, CR⁵R⁶, O, S, NH or NR⁵;

each occurrence of R is, independently, hydrogen or alkyl (e.g., C₁₋₈ alkyl);

each occurrence of R⁵ and R⁶ is independently alkyl (e.g., C₁₋₈ alkyl); and

each occurrence of Z is, independently, a C, N, S, O, or B containing group, a C, N, S, O, or B containing multiple atom chain, or a C, N, S, O, or B containing group multiple atom ring.

In another embodiment, the organic ligand is selected from those listed below:

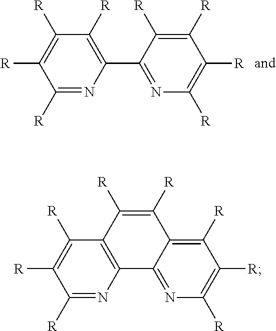

wherein

each occurrence of R is, independently, hydrogen or alkyl (e.g., C₁₋₈ alkyl);

or any two adjacent R groups, together with the connecting carbon atoms, may be linked to form a carbocylic ring (e.g., having 4-7 carbon ring atoms).

In yet another embodiment, the organic ligand is selected from those listed below:

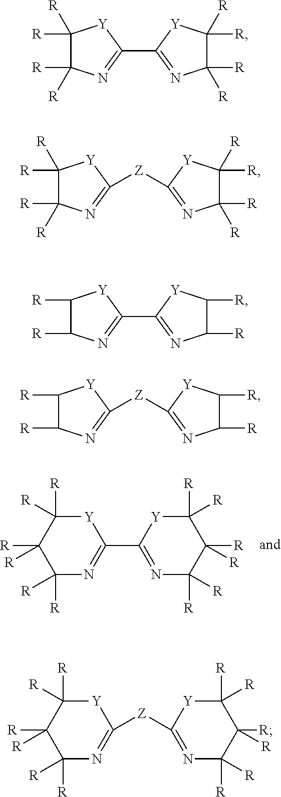

wherein

each occurrence of Y is, independently, CH₂, CHR⁵, CR⁵R⁶, O, S, NH or NR⁵;

each occurrence of R is, independently, hydrogen or alkyl (e.g., C₁₋₈ alkyl);

or any two adjacent R groups, together with the connecting carbon atoms, may be linked to form a carbocylic ring (e.g., having 4-7 carbon ring atoms);

each occurrence of R⁵ and R⁶ is independently alkyl (e.g., C₁₋₈ alkyl); and

each occurrence of Z is, independently, a C, N, S, O, or B containing group, a C, N, S, O, or B containing multiple atom chain, or a C, N, S, O, or B containing group multiple atom ring.

In a preferred embodiment, the organic ligand is selected from diphenylphosphinoethane (dppe), diphenylphosphinopropane (dppp), bis(diphenylphosphino)ethane, 2,2′-bipyridyl, 4,4′-di-tert-butyl-2,2′-bipyridyl (dtbpy), 1,10-phenanthroline, 3,4,7,8-tetramethyl-1,10-phenanthroline, and any combination thereof. In a preferred embodiment, the organic ligand is diphenylphosphinoethane.

In one embodiment, the iridium catalyst is [IrCl(COD)]₂ and the organic ligand is diphenylphosphinoethane.

In another embodiment, the iridium catalyst is [IrCl(COD)]₂ and the organic ligand is a phosphorous containing organic ligand.

In yet another embodiment, the iridium catalyst is [Ir(OMe)(COD)]₂ and the organic ligand is 2,2′-dipyridyl.

In yet another embodiment, the iridium catalyst is [Ir(OMe)(COD)]₂ and the organic ligand is a phosphorous containing organic ligand.

In yet another embodiment, the iridium catalyst is [Ir(OMe)(COD)]₂ and the organic ligand is 1,10-phenanthroline.

In yet another embodiment, the iridium catalyst is [Ir(OMe)(COD)]₂ and the organic ligand is 3,4,7,8-tetramethyl-1,10-phenanthroline.

In one embodiment, the organic ligand is complexed with the catalyst. In one embodiment, the organic ligand and the boron compound are complexed with the catalyst.

In one embodiment, the molar ratio of the compound of Formula (II) to the boron containing compound (for example B₂Pin₂) is from about 0.9 to about 1.2, such as from about 0.95 to about 1.05, from about 0.98 to about 1.02, from about 0.99 to about 1.01 or about 1:1.

In another embodiment, the molar ratio of the compound of Formula (II) to the boron containing compound (for example HBPin) is from about 0.9 to about 2.1, such as from about 0.95 to about 2.05, from about 0.98 to about 2.02, from about 0.99 to about 2.01, or about 2:1.

In one embodiment, the molar ratio of the compound of Formula (II) to the iridium catalyst is from about 100:1 to about 10:1, such as from about 75:1 to about 20:1, or about 70:1 to about 20:1, for example, about 20:1, about 40:1 or about 67:1.

In one embodiment, the amount of iridium catalyst is about 2 to about 10 mole %, such as about 3 to about 6 mole %, or about 5 mole %, based on the number of moles of compound (II).

In another embodiment, the molar ratio of organic ligand to the iridium catalyst is from about 4:1 to about 1:1, such as from about 2.5:1 to about 1.5:1 (e.g., about 2:1).

In another aspect, the present invention relates to a compound of formula (I) prepared by any of the processes described herein.

One embodiment is a composition comprising (a) the compound of formula (I), and (b) less than about 20% by weight (e.g., less than about 10%, about 8%, about 5%, about 4%, about 2%, about 1%, about 0.5%, about 0.2%, about 0.1%, or about 0.05%) of a compound of formula (IV):

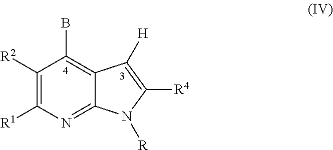

or a salt thereof, based on 100% total weight of compounds (I) and (IV), wherein R, R¹, R², R⁴ and B are as defined above for formula (I).

In a preferred embodiment, the composition contains a compound of formula (IV) in an amount less than about 0.2% by weight (e.g., less than about 0.1% or 0.05%), based on 100% total weight of compounds (I) and (IV).

Another embodiment is a composition comprising (a) the compound of formula (I), and (b) less than about 20% by weight (e.g., less than about 10%, about 8%, about 5%, about 4%, about 2%, about 1%, about 0.5%, about 0.2%, about 0.1%, or about 0.05%) of a multi-borylated product (e.g., a diborylated azaindole such as (i) a 3,6-diborylated azaindole, (ii) an azaindole in which the nitrogen protecting group, R, (such as a para-toluenesulfonyl group) is also borylated, (iii) a 1,3-diborylated azaindole, or (iv) any combination thereof), based on 100% total weight of compound (I) and the multi-borylated product. In a preferred embodiment, the composition contains a multi-borylated product in an amount less than about 0.2% by weight (e.g., less than about 0.1% or 0.05%), based on 100% total weight of compound (I) and the multi-borylated product.

In one preferred embodiment, the ratio of 3-borylated azaindole to 4-borylated azaindole in the product of any of the processes described above is greater than about 90:10, such as greater than about 95:5, greater than about 98:2 or greater than about 99:1.

Any of the processes described above may further comprise converting the compound of formula (I) to a compound of formula (III):

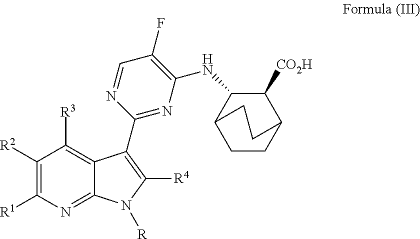

wherein R, R¹, R², R³ and R⁴ are as defined above with respect to formula (I). In a preferred embodiment, R¹, R³ and R⁴ are hydrogen and R² is fluoro. In one embodiment, R is a nitrogen protecting group such as para-toluenesulfonyl.

In another embodiment, any of the processes described above further comprises converting the compound of formula (I) wherein R¹, R³ and R⁴ are hydrogen and R² is fluorine to a compound of the formula (IIIA):

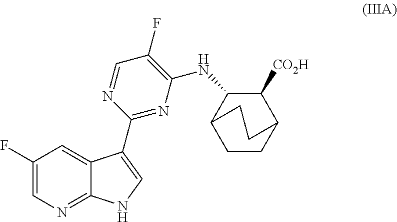 [SMILES: O=C(O)[C@H]1C2CCC(CC2)[C@@H]1Nc1nc(-c2c[nH]c3ncc(F)cc23)ncc1F]

or a salt thereof (e.g., a pharmaceutically acceptable salt thereof).

Suitable processes for conversion of a compound of formula (I) to a compound of formula (III) are described in, for example, International Publication No. WO 2013/019828 and Clark et al., J. Med. Chem., 57, 6668-6678, 2014, each of which is incorporated by reference in its entirety.

For example, a compound of formula (I) where R¹, R³, and R⁴ are hydrogen, R² is fluoro, R is tosyl, and B is 3-(4,4,5,5-tetramethyl-1,3,2-dioxaborolan-2-yl) may be converted to a compound of formula (IIIA) or a salt thereof.

Yet another embodiment is a pharmaceutical composition comprising

(a) (2S,3S)-3-((5-fluoro-2-(5-fluoro-1H-pyrrolo[2,3-b]pyridin-3-yl)pyrimidin-4-yl)amino)bicyclo[2.2.2]octane-2-carboxylic acid or a salt thereof; and

(b) one or more of
(i) 5-fluoro-4-(4,4,5,5-tetramethyl-1,3,2-dioxaborolan-2-yl)-1-tosyl-1H-pyrrolo[2,3-b]pyridine in an amount less than 0.2% by weight, based on the 100% total weight of components (a) and (b)(i),
(ii) 3-((5-fluoro-2-(5-fluoro-1H-pyrrolo[2,3-b]pyridin-4-yl)pyrimidin-4-yl)amino)bicyclo[2.2.2]octane-2-carboxylic acid or a salt thereof in an amount less than 0.2% by weight, based on the 100% total weight of components (a) and (b)(ii),
(iii) methyl 3-((5-fluoro-2-(5-fluoro-1H-pyrrolo[2,3-b]pyridin-4-yl)pyrimidin-4-yl)amino)bicyclo[2.2.2]octane-2-carboxylate or a salt thereof in an amount less than 0.2% by weight, based on the 100% total weight of components (a) and (b)(iii), and
(iv) a compound of formula (IB):

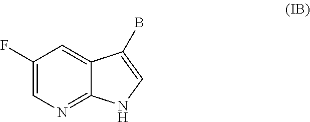

or a salt thereof, wherein

B is a boryl containing group where the boron atom of the group is attached to the carbon ring atom at the 3-position of the azaindole,

wherein component (b)(iv) is present in an amount less than 0.2% by weight, based on the 100% total weight of components (a) and (b)(iv).

The pharmaceutical composition can be, for example, in the form of an oral dosage form, such as a tablet, capsule, or oral solution.

Yet another embodiment is a method for preparing 2-amino-3-bromo-5-fluoropyridine comprising the step of reacting 2-amino-5-fluoropyridine with bromine in sulfuric acid. The 2-amino-3-bromo-5-fluoropyridine may be subsequently converted to 2-amino-3-((trimethylsilyl)ethynyl)-5-fluoropyridine, which can be can be cyclized and tosylated to form 5-fluoro-1-tosyl-7-azaindole. The 5-fluoro-1-tosyl-7-azaindole can be converted to compounds of formulas (I), (III), and (IIIA) by the methods described herein.

### DETAILED DESCRIPTION OF THE INVENTION

### Definitions

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl).

The term “alkenyl ” refers to an aliphatic hydrocarbon group containing a carbon-carbon double bond and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl.

The term “alkynyl” refers to a straight or branched chain hydrocarbyl radical having at least one carbon-carbon triple bond, and having in the range of about 2 up to 12 carbon atoms (with radicals having in the range of about 2 to 10 carbon atoms presently being preferred) e.g., ethynyl, propynyl, and butnyl.

The term “alkoxy” denotes an alkyl group as defined above attached via an oxygen linkage to the rest of the molecule. Representative examples of those groups are —OCH₃ and —OC₂H₅.

The term “cycloalkyl” denotes a non-aromatic mono or multicyclic ring system of about 3 to 12 carbon atoms such as cyclopropyl, cyclobutyl, cyclopentyl, and cyclohexyl. Non-limiting examples of multicyclic cycloalkyl groups include perhydronapththyl, adamantly, norbornyl groups (bridged cyclic group), or spirobicyclic groups e.g. spiro (4,4) non-2-yl.

The term “cycloalkylalkyl” refers to a cyclic ring-containing radical containing in the range of about 3 up to 8 carbon atoms directly attached to an alkyl group which is then attached to the main structure at any carbon in the alkyl group, such as cyclopropylmethyl, cyclobuyylethyl, and cyclopentylethyl.

The term “aryl” refers to an aromatic radical having in the range of 6 up to 20 carbon atoms such as phenyl, naphthyl, tetrahydronapthyl, indanyl, and biphenyl.

The term “arylalkyl” refers to an aryl group as defined above directly bonded to an alkyl group as defined above, e.g., —CH₂C₆H₅, and —C₂H₅C₆H₅.

The term “heterocyclyl” refers to a non-aromatic 3 to 15 member ring radical which, consists of carbon atoms and at least one heteroatom selected from the group consisting of nitrogen, phosphorus, oxygen and sulfur. For purposes of this invention, the heterocyclyl radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized.

The term “heteroaryl” refers to an optionally substituted 5-14 member aromatic ring having one or more heteroatoms selected from N, O, and S as ring atoms. The heteroaryl may be a mono-, bi- or tricyclic ring system. Examples of such heteroaryl ring radicals includes but are not limited to oxazolyl, thiazolyl imidazolyl, pyrrolyl, furanyl, pyridinyl, pyrimidinyl, pyrazinyl, benzofuranyl, indolyl, benzothiazolyl, benzoxazolyl, carbazolyl, quinolyl and isoquinolyl. The heteroaryl ring radical may be attached to the main structure at any heteroatom or carbon atom.

Examples of such “heterocyclyl” or “heteroaryl” radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide thiamorpholinyl sulfone, dioxaphospholanyl, oxadiazolyl, chromanyl, and isochromanyl.

The term “heteroarylalkyl” refers to a heteroaryl ring radical as defined above directly bonded to an alkyl group. The heteroarylalkyl radical may be attached to the main structure at any carbon atom from the alkyl group.

The term “heterocyclylalkyl” refers to a heterocylic ring radical as defined above directly bonded to an alkyl group. The heterocyclylalkyl radical may be attached to the main structure at a carbon atom in the alkyl group.

The term “substituted” unless otherwise specified refers to substitution with any one or any combination of the following substituents: hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted aryl, substituted or unsubstituted heteroaryl, substituted heterocyclylalkyl ring, substituted or unsubstituted heteroarylalkyl, substituted or unsubstituted heterocyclic ring, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —SO₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, and —ONO₂, wherein R^{x}, R^{y} and R^{z} in each of the above groups can be hydrogen atom, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, substituted or unsubstituted heterocyclic ring, or any two of R^{x}, R^{y} and R^{z} may be joined to form a substituted or unsubstituted saturated or unsaturated 3-10 member ring, which may optionally include heteroatoms which may be same or different and are selected from O, NR^{X} or S. The substituents in the aforementioned “substituted” groups cannot be further substituted. For example, when the substituent on “substituted alkyl” is “substituted aryl”, the substituent on “substituted aryl” cannot be “substituted alkenyl”. Substitution or the combination of substituents envisioned by this invention are preferably those resulting in the formation of a stable compound.

The term “protecting group” refers to a substituent that is employed to block or protect a particular functionality. Other functional groups on the compound may remain reactive. For example, a “nitrogen protecting group” is a substituent attached to a nitrogen atom that blocks or protects the nitrogen functionality in the compound. Suitable nitrogen protecting groups include, but are not limited to, acetyl, trifluoroacetyl, tert-butoxycarbonyl, (benzyloxy)carbonyl, N,N-dimethylaminosulfonyl, N,N-dimethylcarboxamide, para-toluenesulfonyl and 9H-fluoren-9-ylmethyloxycarbonyl. For a general description of protecting groups and their use, see, e.g., T. W. Greene, Protective Groups in Organic Synthesis, John Wiley & Sons, New York, 1991.

All the stereoisomers of compounds described herein are within the scope of this invention. Racemic mixtures are also encompassed within the scope of this invention. Therefore, single stereochemical isomers as well enantiomeric, diastereoisomeric and geometric (or conformational) mixtures of the present compounds fall within the scope of the invention.

The term “stereoisomer” refers to compounds, which have identical chemical composition, but differ with regard to arrangement of the atoms and the groups in space. These include enantiomers, diastereomers, geometrical isomers, atropisomer or conformational isomers.

### Experimental

### EXAMPLE 1

### Synthesis of 3-BPin-5-bromo-7-azaindole

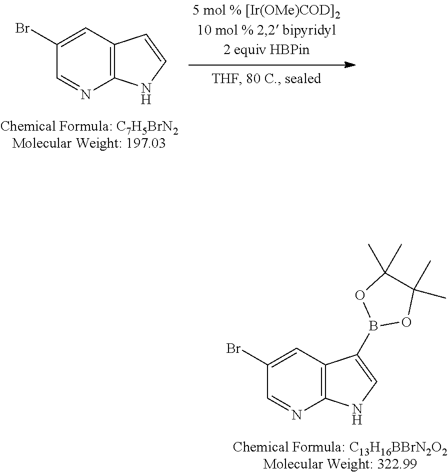 [SMILES: Brc1cnc2[nH]ccc2c1 ; CC1(C)OB(c2c[nH]c3ncc(Br)cc23)OC1(C)C]

5-fluoro-7-azaindole (1 g) and THF (10 mL) were added to a small screw-top vial fitted with a septum, argon inlet and exit needle. The flask was sparged with argon for ˜10 minutes. The iridium catalyst [Ir(OMe)COD]₂ (0.168 mg) and 2,2′-bipyridyl (0.080 mg) were added as solids and the flask was covered with the septum and sparged with argon again for ˜10-15 minutes. When the catalyst was added, the reaction turned a dark red/purple. The argon was then turned off, and HBPin (1.5 mL) was added via syringe. The reaction bubbled, releasing hydrogen. The hydrogen was allowed to bubble out through the bubbler outlet and once bubbling stopped, the reaction was capped and placed in an oil bath heated to 80° C.

After approx. 20 hours, the flask was allowed to cool to room temperature and a sample was pulled from the reaction flask for HPLC analysis. The reaction was then quenched with methanol (10 mL) and allowed to stir for ˜5 minutes before it was concentrated in vacuo to afford a dark residue (2.44 g). The residue was dissolved in methyl t-butyl ether (MTBE) (50 mL) and filtered through a silica plug (20 g, 150 mL frit). The cake was washed with MTBE (3×20 mL) and the filtrate was collected and concentrated in vacuo to afford 1.5 g of an off-white solid. The solid residue was taken up in 10 mL of isopropanol (IPA) and heated until it dissolved. The flask was allowed to cool to room temperature, at which point some crystals had precipitated out of solution. The flask was placed in the freezer overnight to afford white crystals.

The crystals were filtered in vacuo, washed with cold hexanes, and dried on a rotovap (yield: 0.5 g). The crystals were taken up again in hexanes (10 mL) and heated to reflux, but the crystals would not dissolve in the hexanes. Thus, the hexanes were removed on the rotovap, and the crystals were taken up in IPA (10 mL) and heated to reflux until the crystals dissolved. The flask was allowed to cool to room temperature, and then placed in the freezer overnight to crystallize, affording 300 mg (7.8%) of product.

### EXAMPLE 2

### Synthesis of 3-BPin-5-bromo-N-tosyl-7-azaindole

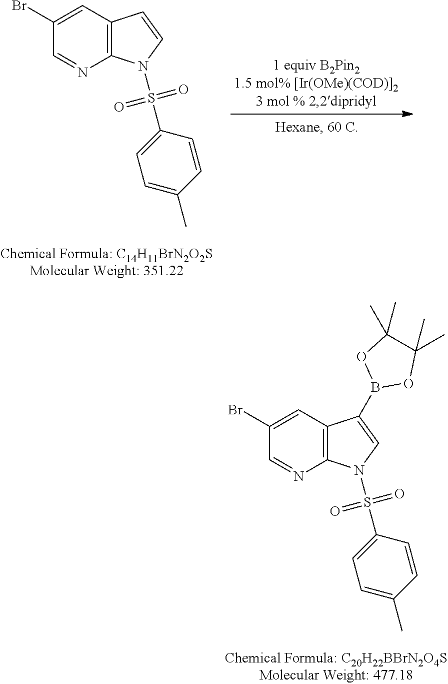 [SMILES: Cc1ccc(S(=O)(=O)n2ccc3cc(Br)cnc32)cc1 ; Cc1ccc(S(=O)(=O)n2cc(B3OC(C)(C)C(C)(C)O3)c3cc(Br)cnc32)cc1]

A 250 mL 1-neck round bottom flask equipped with a thermocouple and argon inlet was sparged with argon for 15 minutes. 5-bromo-N-tosyl-7-azaindole (4.0 g), B₂Pin₂ (2.90 g), [Ir(OMe)COD]₂ (0.114 g), 2,2′bipyridyl (0.054 g) and hexane (50 mL) were then added. The flask was again inerted with 3 vacuum purges. The resulting brown slurry was then heated to 60° C. incrementally (setpoints: 45° C., 55° C., 58° C. and 60° C.) and stirred overnight.

After 15 hours at 60° C., HPLC of the reaction mixture indicated 3.0% starting material remaining and 94% product. After 18 hours at 60° C., HPLC of the reaction mixture indicated 2.7% starting material and 5% product.

The slurry was then cooled to room temperature and vacuum filtered. The solids were recombined with the mother liquor and concentrated to afford a solid. The solid was dissolved in dichloromethane (50 mL) and filtered through silica (5 g on a 60 mL frit). The plug was washed with dichloromethane (2×50 mL). The filtrate and washes were combined and concentrated by rotovap to approx. 50 mL. Hexane (50 mL) was added and the solution was again concentrated to approx. 50 mL. Hexane (50 mL) was again added and the solution was concentrated. Product that “bumped” was rinsed back into the flask with dichloromethane. The solution was concentrated to approx. 50 mL and hexane (35 mL) was added. The solution was then concentrated to approx. 50 mL. The slurry was vacuum filtered and the solids were washed with cold hexane, then dried by rotovap, to afford 4.7 g (88.7% of product. HPLC indicated approx. 97% purity.

### EXAMPLE 3

### Synthesis of 3-BPin-5-fluoro-N-tosyl-7-azaindole

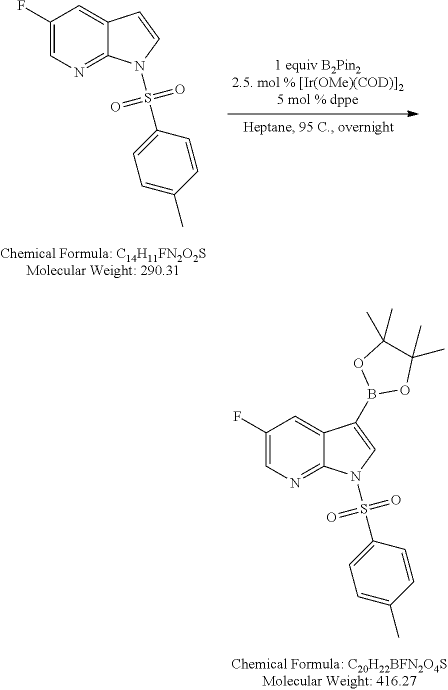 [SMILES: Cc1ccc(S(=O)(=O)n2ccc3cc(F)cnc32)cc1 ; Cc1ccc(S(=O)(=O)n2cc(B3OC(C)(C)C(C)(C)O3)c3cc(F)cnc32)cc1]

A 1 L 3-necked flask equipped with mechanical stirring, argon inlet, thermocouple, and heating mantle was sparged with argon for 15 minutes. 5-fluoro-N-tosyl-7-azaindole (50.0 g), B₂Pin₂ (43.7 g), [IrClCOD]₂ (2.89 g), dppe (3.43 g), and heptane (500 mL) were then added to the flask. The resulting slurry was then heated to 95 C for 53 hours with stirring.

The slurry was then cooled to room temperature and the solids were collected by vacuum filtration, washed with cold hexane, and dissolved in dichloromethane (450 mL). The solution was filtered through silica (100 g on a 600 mL frit) and the plug was washed with 5×100 mL dichloromethane. The filtrate and washes were combined and concentrated by rotovap. Hexane (400 mL) was then added and the solution was concentrated to approx 200 mL. The slurry was then filtered and the solids washed with cold hexane, dried by rotovap to afford 56.97 g (79.6%) of product. HPLC indicated a purity of >99%.

### EXAMPLE 4

### Synthesis of N-Boc-3-BPin-5-fluoro-7-azaindole

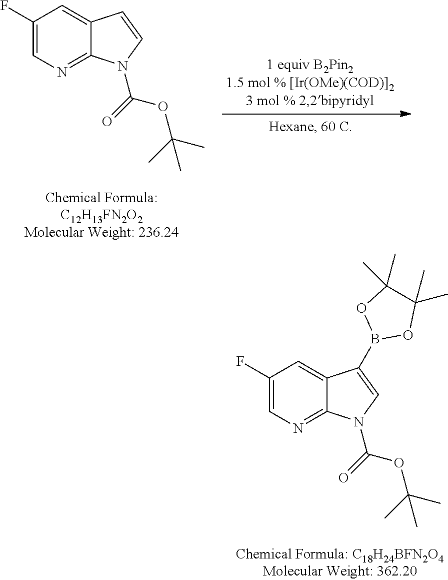 [SMILES: CC(C)(C)OC(=O)n1ccc2cc(F)cnc21 ; CC(C)(C)OC(=O)n1cc(B2OC(C)(C)C(C)(C)O2)c2cc(F)cnc21]

N-Boc-5-fluoro-7-azaindole (5.0 g), B₂Pin₂ (5.38 g), and hexane (50 mL) were added to a 250 mL 2-neck round bottom equipped with a condenser, magnetic stirring, heating mantle and nitrogen inlet. A colorless solution resulted with stirring. The flask was sparged with 3 nitrogen/vacuum cycles. The iridium catalyst [Ir(OMe)COD]₂ (0.21 g) and 2,2′-bipyridyl (0.10 g) were added as solids and another nitrogen/vacuum cycle was used to inert the flask. The resulting black solution was heated to 60° C. After 1 hour, TLC (eluting with DCM) of the reaction solution indicated that no starting material remained. The solution was cooled to room temperature and filtered through silica (10 g on a 60 mL frit). The plug was washed with dichloromethane (4×100 mL). The fractions were combined and concentrated by rotovap until a precipitate began to form. Dichloromethane was then added until a solution resulted and hexane (50 mL) was added. The solution was concentrated cold <25° C. to approx. 50 mL. Hexane (50 mL) was added and the solution was concentrated to approx. 75 mL. The resulting white solids were collected by vacuum filtration, washed with cold hexane and dried by rotovap, to afford 4.6 g (60.5%) of product.

### EXAMPLE 5

### Synthesis of 3-BPin-7-azaindole

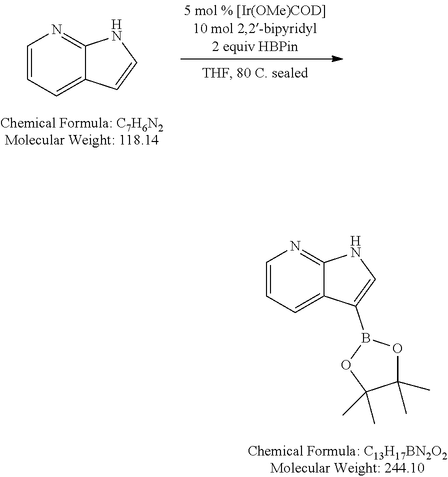 [SMILES: c1cnc2[nH]ccc2c1 ; CC1(C)OB(c2c[nH]c3ncccc23)OC1(C)C]

7-azaindole (5.0 g) and THF (50 mL) were added to an argon-inert small screw-top vial fitted with a septum, argon inlet and bubbler outlet. The flask was sparged with argon. The iridium catalyst [Ir(OMe)COD]₂ (1.4 g) and 2,2′bipyridyl (1.4 g) were then added and the flask was again sparged with argon. The HBPin (12. 3 mL) was added by syringe and gas evolution was observed. The screwtop was sealed and the vial was placed in an oil bath and heated to 80° C. for 16 hours.

The vessel was then allowed to cool to room temperature. The cap was removed and sampled while under an argon stream. The reaction appeared to stall at 50% completion. The cap was removed and 20 mL of methanol was added with visible degassing. The combined reaction solution was concentrated to an oil by rotovap (17.28 g). The crude product was dissolved in 50 mL of MTBE and filtered through 50 g of silica. The plug was washed with 3×50 mL of MTBE and the filtrate was concentrated by rotovap (13 g of crude product). The crude product was dissolved in 13 mL of refluxing IPA, cooled to room temperature, and placed in the freezer. No crystals were observed.

### EXAMPLE 6

### Synthesis of 3-BPin-5-fluoro-7-azaindole

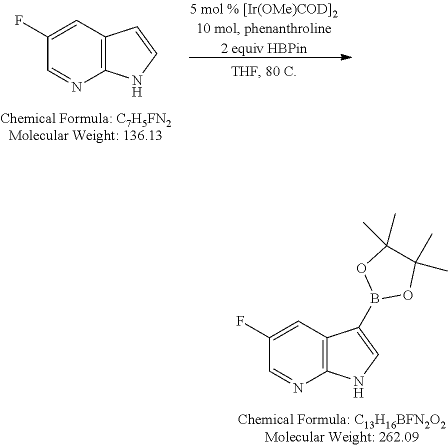 [SMILES: Fc1cnc2[nH]ccc2c1 ; CC1(C)OB(c2c[nH]c3ncc(F)cc23)OC1(C)C]

5-fluoro-7-azaindole (1.0 g) and THF (10 mL) were added to an argon-inert small screw-top vial fitted with a septum, argon inlet and bubbler outlet. The flask was sparged with argon. The iridium catalyst [Ir(OMe)COD]₂ (0.24 g) and 3,4,7,8-tetramethyl-1,10-phenanthroline (0.11 g) were then added and the flask was again sparged with argon. HBPin (2.13 mL) was added by syringe and gas evolution was observed. The screwtop was sealed and the vial was placed in an oil bath and heated to 80° C. overnight.

The vial was then removed from the oil bath and allowed to cool to room temperature. The reaction was quenched by the addition of methanol (20 mL, very little gas evolution noted). The solution was then concentrated by rotovap to afford a dark oil (3.57 g). The oil was dissolved in MTBE (50 mL) and filtered though silica. The plug was washed with MTBE (4×25 mL) and the clear, yellow filtrate was concentrated by rotovap to an oil (2.7 g).

Upon standing overnight, solids precipitated out of the crude oil. The oil was then dissolved in refluxing hexane (3 mL, ˜1 mL/g) and the solution was allowed to cool to room temperature then placed in the freezer.

The resulting white solids were collected by vacuum filtration, washed three times with cold hexane, and dried by rotovap (0.58 g crude product). HPLC indicated 92.2% purity. The crude solids were dissolved in refluxing IPA (1.2 mL, ˜2 mL/g) and the resulting yellow solution was allowed to cool to room temperature (during which time crystals precipitated) then placed in the freezer. The resulting crystals were collected by vacuum filtration, washed three times with cold hexane (3×), and dried by rotovap to afford 0.33 g (17.1%) of product.

While the present invention is described herein with reference to illustrated embodiments, it should be understood that the invention is not limited hereto. Those having ordinary skill in the art and access to the teachings herein will recognize additional modifications and embodiments within the scope thereof.

All patents, patent applications, government publications, government regulations, and literature references cited in this specification are hereby incorporated herein by reference in their entirety. In case of conflict, the present description, including definitions, will control.

1. A process for preparing a compound of formula (I): 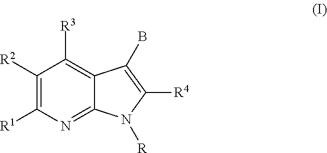
wherein
R is hydrogen or a nitrogen protecting group;
R¹, R², R³ and R⁴ are each independently selected from hydrogen, halogen, hydroxyl, nitro, amino, substituted or unsubstituted alkylamino, substituted or unsubstituted dialkylamino, cyano, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted cycloalkylalkyl, substituted or unsubstituted heterocyclylalkyl, substituted or unsubstituted heteroaryl, substituted or unsubstituted arylalkyl, and substituted or unsubstituted heteroarylalkyl; and
B is a boryl containing group where the boron atom of the group is attached to the carbon ring atom at the 3-position of the azaindole,
the process comprising reacting a compound of formula (II) 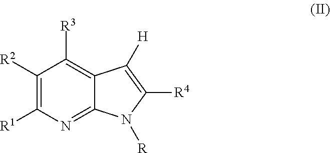
with a boron containing compound in the presence of an organic ligand and a catalyst selected from iridium catalysts, rhodium catalysts, ruthenium catalysts, and any combination thereof, with the proviso that (i) the iridium catalyst is not [Ir(OMe)(COD)]₂, (ii) R is not Boc, (iii) R² is a halogen, or (iv) any combination thereof.

2. The process of claim 1, wherein R is selected from the group consisting of tert-butoxycarbonyl, (benzyloxy)carbonyl, N,N-dimethylaminosulfonyl, N,N-dimethylcarboxamide, para-toluenesulfonyl, 9H-fluoren-9-ylmethyloxycarbonyl, benzenesulfonyl, t-butyl, t-butyldimethylsilane and a dialkoxyborane.

3. The process of claim 1, wherein R is para-toluenesulfonyl.

4. The process of claim 1, wherein R¹, R³ and R⁴ are hydrogen and R² is halogen.

5. The process of claim 1, wherein R² is fluoro.

6. The process of claim 1, wherein R² is fluoro and R is hydrogen.

7. The process of claim 1, wherein B is 4,4,5,5-tetramethyl-1,3,2-dioxaborolan-2-yl.

8. The process of claim 1, wherein the iridium catalyst is selected from the group consisting of [Ir(OMe)(COD)]₂, [Ir(Cl)(COD)]₂, (COD) (η⁵-indenyl)Ir, and combinations thereof, wherein COD is 1,5-cyclooctadiene.

9. The process of claim 1, wherein the iridium catalyst is [Ir(Cl)(COD)]₂.

10. The process of claim 1, wherein the organic ligand is selected from the group consisting of diphenylphosphinoethane, bis(diphenylphosphino)ethane, 2,2′-bipyridyl, 4,4′-di-tert-butyl-2,2′-bipyridyl (dtbpy), 1,10-phenanthroline, 3,4,7,8-tetramethyl-1,10-phenanthroline, and combinations thereof.

11. The process of claim 1, wherein the boron containing compound is selected from HBPin, diborane, B₂(Cat)₂, and B₂Pin_{2.}

12. The process of claim 1, wherein the iridium catalyst is [Ir(Cl)(COD)]₂ and the organic ligand is diphenylphosphinoethane.

13. The process of claim 1, wherein, the reaction is conducted in a solvent selected from the group consisting of aliphatic hydrocarbons, ethers, chlorinated alkanes, and any combination thereof.

14. The process of claim 1, wherein the reaction is conducted in a solvent selected from the group consisting of hexane, heptane, tetrahydrofuran, and any combination thereof.

15. The process of claim 1, further comprising converting the compound of formula (I) to a compound of formula (III): 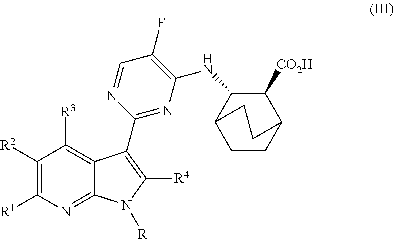
or a salt thereof.

16. The process of claim 1, further comprising converting the compound of formula (I) to a compound of formula (IIIA): 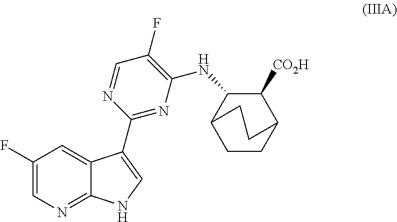 [SMILES: O=C(O)[C@H]1C2CCC(CC2)[C@@H]1Nc1nc(-c2c[nH]c3ncc(F)cc23)ncc1F]
or a salt thereof.

17. The compound of formula (I) prepared by the process of claim 1.

18. A compound of formula (IA): 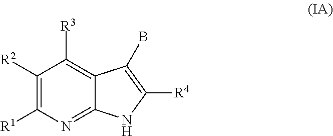
or a salt thereof, wherein
R¹, R², R³ and R⁴ are each independently selected from hydrogen, halogen, hydroxyl, nitro, amino, substituted or unsubstituted alkylamino, substituted or unsubstituted dialkylamino, cyano, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted cycloalkylalkyl, substituted or unsubstituted heterocyclylalkyl, substituted or unsubstituted heteroaryl, substituted or unsubstituted arylalkyl, and substituted or unsubstituted heteroarylalkyl; and
B is a boryl containing group where the boron atom of the group is attached to the carbon ring atom at the 3-position of the azaindole.

19. A method for preparing 2-amino-3-bromo-5-fluoropyridine comprising the step of reacting 2-amino-5-fluoropyridine with bromine in sulfuric acid.

20. The method of claim 19, further comprising converting 2-amino-3-bromo-5-fluoropyridine to 2-amino-3-((trimethylsilyl)ethynyl)-5-fluoropyridine.

21. The method of claim 19, further comprising
cyclizing and tosylating 2-amino-3-((trimethylsilyl)ethynyl)-5-fluoropyridine to 5-fluoro-1-tosyl-7-azaindole;
converting 5-fluoro-1-tosyl-7-azaindole to 5-fluoro-3-(4,4,5,5-tetramethyl-1,3,2-dioxaborolan-2-yl)-1-tosyl-1H-pyrrolo[2,3-b]pyridine;
converting 5-fluoro-3-(4,4,5,5-tetramethyl-1,3,2-dioxaborolan-2-yl)-1-tosyl-1H-pyrrolo[2,3-b]pyridine to (2S,3S)-3-((5-fluoro-2-(5-fluoro-1H-pyrrolo[2,3-b]pyridin-3-yl)pyrimidin-4-yl)amino)bicyclo[2.2.2]octane-2-carboxylic acid; and
optionally converting (2S,3S)-3-((5-fluoro-2-(5-fluoro-1H-pyrrolo[2,3-b]pyridin-3-yl)pyrimidin-4-yl)amino)bicyclo[2.2.2]octane-2-carboxylic acid to a salt thereof.

22. A pharmaceutical composition comprising
(a) (2S,3S)-3-((5-fluoro-2-(5-fluoro-1H-pyrrolo[2,3-b]pyridin-3-yl)pyrimidin-4-yl)amino)bicyclo[2.2.2]octane-2-carboxylic acid or a salt thereof; and
(b) one or more of
(i) 5-fluoro-4-(4,4,5,5-tetramethyl-1,3,2-dioxaborolan-2-yl)-1-tosyl-1H-pyrrolo[2,3-b]pyridine in an amount less than 0.2% by weight, based on the 100% total weight of components (a) and (b)(i),
(ii) 3-((5-fluoro-2-(5-fluoro-1H-pyrrolo[2,3-b]pyridin-4-yl)pyrimidin-4-yl)amino)bicyclo[2.2.2]octane-2-carboxylic acid or a salt thereof in an amount less than 0.2% by weight, based on the 100% total weight of components (a) and (b)(ii),
(iii) methyl 3-((5-fluoro-2-(5-fluoro-1H-pyrrolo[2,3-b]pyridin-4-yl)pyrimidin-4-yl)amino)bicyclo[2.2.2]octane-2-carboxylate or a salt thereof in an amount less than 0.2% by weight, based on the 100% total weight of components (a) and (b)(iii), and
(iv) a compound of formula (TB): 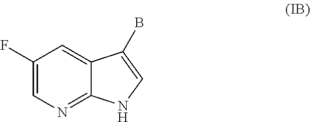
or a salt thereof, wherein
R¹, R², R³ and R⁴ are each independently selected from hydrogen, halogen, hydroxyl, nitro, amino, substituted or unsubstituted alkylamino, substituted or unsubstituted dialkylamino, cyano, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted heterocyclyl, substituted or unsubstituted aryl, substituted or unsubstituted cycloalkylalkyl, substituted or unsubstituted heterocyclylalkyl, substituted or unsubstituted heteroaryl, substituted or unsubstituted arylalkyl, and substituted or unsubstituted heteroarylalkyl; and
B is a boryl containing group where the boron atom of the group is attached to the carbon ring atom at the 3-position of the azaindole,
wherein component (b)(iv) is present in an amount less than 0.2% by weight, based on the 100% total weight of components (a) and (b)(iv).

23. The pharmaceutical composition of claim 22, wherein the pharmaceutical composition is in the form of an oral dosage form.
